(19) United States
(12) Patent Application Publication
(10) Pub. No.: US 2014/0107593 A1
(43) Pub. Date: Apr. 17, 2014

(54) FENESTRATED NEEDLE FOR DELIVERING THERAPEUTIC REAGENTS INTO SOFT TISSUE

(71) Applicant: SpineSmith Partners, L.P., Austin, TX (US)

(72) Inventors: Jizong Gao, Cedar Park, TX (US); Richard J. Kana, Lexington, TX (US)

(73) Assignee: SpineSmith Partners, L.P., Austin, TX (US)

(21) Appl. No.: 14/055,720

(22) Filed: Oct. 16, 2013

Related U.S. Application Data
(60) Provisional application No. 61/714,366, filed on Oct. 16, 2012.

(51) Int. Cl. A61M 5/158

(52) U.S. Cl. CPC A61M 5/158
USPC 604/272

(57) ABSTRACT

An embodiment of the claimed invention is a therapeutic delivery device having a plurality of fenestrations located around the circumference of its distal portion and an opening at the tip. The opening at the tip and the plurality of fenestrations reduce the injection resistance, making it easy to use, and ensure an even distribution of the injected material at the recipient location.

### CROSS-REFERENCES TO RELATED APPLICATIONS

This Application claims the benefit under 35 U.S.C. §119(e) of U.S. Provisional Patent Application No. 61/714,366 filed Oct. 16, 2012, which is incorporated herein by reference in its entirety as if fully set forth herein.

### BACKGROUND OF THE INVENTION

The standard way to deliver therapeutic reagents into soft tissue is through the use of needle injection. Currently, the injection needle that is used for this purpose typically has a single opening at its tip. The disadvantages for such an injection needle include high resistance during the injection, non-even distribution of the injected material and potential blockages of the opening on the tip. When discography or disc-injection is performed in the spine surgery field, a needle (injection needle) is inserted first followed by a second needle (inducer needle) that is smaller in diameter but longer than the first needle is inserted into the first needle to reach to the center of the nucleus pulpous. In currently available systems, the lures on the two needles do not engage each other by a locking mechanism, which can cause injection error and operation difficulty.

Therefore, it would be desirable to design and develop a fenestrated needle that overcomes the disadvantages of presently available fenestrated needle systems.

### SUMMARY OF THE INVENTION

An embodiment of the claimed invention is directed to a therapeutic delivery device for delivering therapeutics comprising: a first needle comprising at least one hole or passage for receiving a second needle or stylet; a connector at the proximal end of the first needle, wherein the connector removably connects the second needle or stylet to the first needle, and further wherein the first needle comprises more than one fenestration at its distal end.

It is an object of the claimed invention to provide lower injection resistance; even distribution of the injected material at the recipient site; reduced possibility for tissue blockage of the needle; and a dual lure connector at the proximal end of the needle that enables stable connection with both the inducer needle and the syringes.

### BRIEF DESCRIPTION OF THE DRAWINGS

FIG. 1A shows a first (injection) needle in accordance with an embodiment of the invention;

FIG. 1B shows a second (inducer) needle/stylus in accordance with an embodiment of the invention;

FIG. 2A shows a therapeutic delivery device comprising an assembled first needle and second needle in accordance with an embodiment of the invention; and

FIG. 2B shows a magnified view of the distal end of the therapeutic delivery device of FIG. 2A.

### DETAILED DESCRIPTION OF EXEMPLARY EMBODIMENTS

An embodiment of the claimed invention is a therapeutic delivery device having a plurality of fenestrations located around the circumference of its distal portion and an opening at the tip. The opening at the tip and the plurality of fenestrations reduce the injection resistance, making it easy to use, and ensure an even distribution of the injected material at the recipient location.

Another embodiment of the claimed invention is directed to a therapeutic delivery device for delivering therapeutics comprising: a first needle comprising at least one conduit or passage for receiving a second needle or stylet; a connector at the proximal end of the first needle, wherein the connector removably connects the second needle or stylet to the first needle, and further wherein the first needle comprises more than one fenestration at its distal end.

In an embodiment of the claimed invention, the proximal end of the injection needle has a dual luer connector comprising a male end and a female end. The female luer of the duel luer connector connects with the male luer of the inducer needle/stylet by a locking mechanism. During the needle-in-needle injection procedure, this mechanism ensures the stability of the injection needle and reduces the possibility of tissue damage due to unwanted motion of the needles. As further detailed below, the fenestrations in the injection needle are also designed to prevent soft tissue from blocking the fenestration during the advancement of the needle within the soft tissue.

In an embodiment of the invention, fenestrations at the distal end of the injection needle in accordance with the claimed invention, have a special arrangement to prevent blockage. In certain embodiments, the orientation of the fenestrations is 15 degrees to 30 degrees with respect to the long axis of the needle with the opening angled toward the proximal end. This arrangement reduces the blockage of the fenestration by soft tissue when the needle is inserted and advanced through soft tissue. In other words, the angling of the fenestration openings in a direction (proximal) that is opposite to the direction (distal) of the advancement of the needle prevents the fenestrations from grasping and snagging the soft tissue when the needle is advanced into the soft tissue during use.

In another embodiment of the invention, a dual luer connector is located at the proximal end of the injection needle. The dual luer connector has a distally located male luer and a proximally located female luer. The distally located male luer is capable of connecting with a female luer on a syringe. The proximally located female luer is capable of connecting with the male luer of the second/inducer needle. When the proximally located female luer of the injection needle is connected to the male luer of inducer needle, a locking mechanism is created which holds the needles in place during use. This locking arrangement of the dual luers of the injection needle and the inducer needle is particularly essential when performing the disc injection using the “needle-in-needle” method.

In certain embodiments of the invention, the distal end of the injection needle has a triangle opening which holds and fits the diamond-shape sharpened end of the stylet. Each wing of the triangle opening contains at least one fenestration.

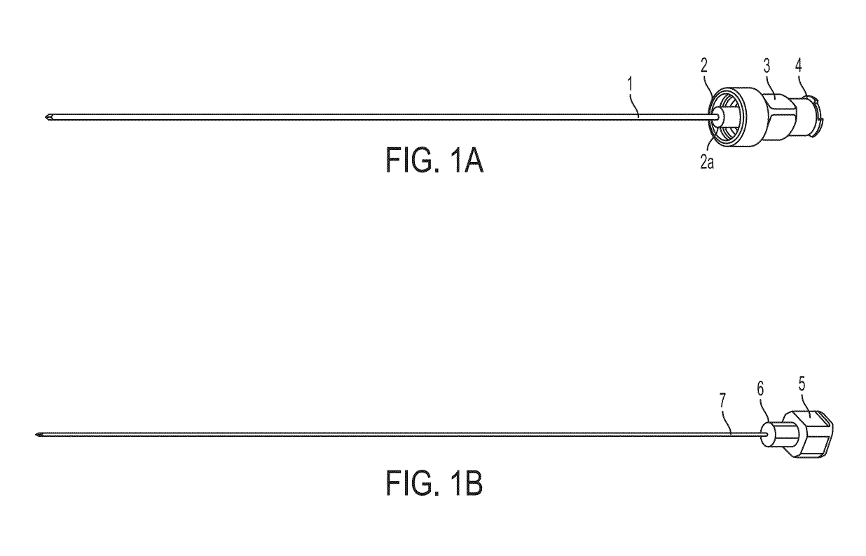

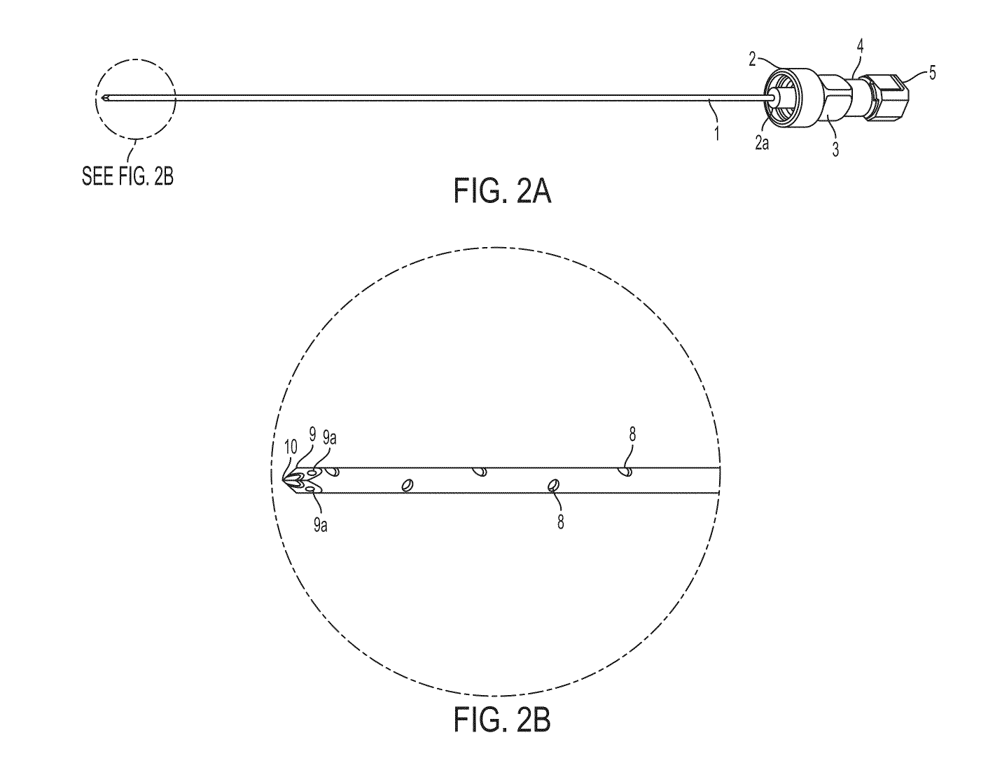

Embodiments of the inventions are further described below accompanied by the drawings set forth in FIGS. 1A, 1B, 2A and 2B.

FIG. 1A shows a first needle subassembly or an injection needle subassembly in accordance with an embodiment of the invention. An injection needle 1 is shown in FIG. 1A. The injection needle 1 is connected to the distal end 2 of a duel luer connector 3. The dual luer connector 3 has a male luer 2a at its distal end. The duel luer connector comprises a female luer 4 at its proximal end. The needle 1 contains a passage or conduit within the needle, which can be accessed via the female luer 4. In other words, the conduit or passage within needle 1 is aligned with conduits/passages located within the male luer 2a, duel luer connector 3 and female luer 4. This design facilitates access to the passage/conduit of needle 1 from the female luer 4.

As seen in FIG. 1B, an embodiment of the invention is directed to a second needle subassembly. An inducer needle or stylet 7 is shown in FIG. 1B. At its proximal end, the needle 7 is connected to a connector 5 via a male luer 6. In an embodiment of the invention, the needle 7 is capable of being inserted into the conduit/passage of needle 1. This insertion occurs by introducing the distal end of needle 7 into the female luer 4 and pushing the length of needle 7 through the length of the conduit/passage of needle 1. In certain embodiments of the invention, upon insertion of the needle 7 into the passage/conduit of needle 1, the male luer 6 is engaged/connected to the female luer 4 to form a locking mechanism. In certain embodiments of the invention, the male luer is removably engaged/connected to the female luer 4 within the locking mechanism.

FIG. 2A shows an assembled therapeutic delivery device in accordance with an embodiment of the invention. As shown in FIG. 2A, the second needle subassembly is fully inserted into the passage/conduit of needle 1, such that the needle 7 is fully inserted and seated within the passage/conduit of needle 1, and the male luer 6 is engaged/connected to the female luer 4 to form a locking mechanism. In the assembled state as set forth in FIG. 2A, the connector 5 contacts the proximal end of the female luer 4.

FIG. 2B shows an exploded view of the distal portion of the assembled therapeutic delivery device in FIG. 2A. As seen in FIG. 2B, the distal portion of the needle 1 of the therapeutic delivery device comprises a plurality of fenestrations 8 around the circumference of the distal portion of the cylinder of the needle 1. The fenestrations 8 are angled with respect to the longitudinal axis of needle such that the fenestration openings are angled toward the proximal end, i.e., in an opposite direction to the direction of the insertion of the therapeutic delivery device into a subject when in use (see arrow). The angled direction of the fenestrations is indicated in FIG. 2B by dotted lines that extend from the fenestration 8 opening to the interior of the needle 1. In certain embodiments of the invention, the angle of the fenestrations 8 relative to the longitudinal axis of the injection needle 1, ranges from 15° to 30°. The direction of insertion of the therapeutic delivery device into a subject during use is indicated by the arrow in FIG. 2B.

As further shown in FIG. 2B, the distal portion of the needle 1 has a triangle-shaped opening 9 at its tip that holds the pyramid-shaped tip 10 of the second needle/stylus 7, when the second needle subassembly is assembled with the first needle subassembly. The triangle opening 9 further comprises at least one hole or fenestration 9a on either side/wing of the opening 9.

An embodiment of the invention is directed to a kit for the injection of therapeutic material. In certain embodiments, the kit comprises syringes having varying volumetric ratios, cartridges for the syringes, a connector for multiple syringes, a blender to connect multiple syringes and one or more therapeutic delivery devices as described and claimed herein.

In the preceding detailed description, the invention is described with reference to specific exemplary embodiments thereof and locations of use within the spine. Various modifications and changes may be made thereto without departing from the broader spirit and scope of the invention as set forth in the claims. The specification and drawings are, accordingly, to be regarded in an illustrative rather than a restrictive sense.

What is claimed is:

1. A therapeutic delivery device for delivering therapeutics comprising: a first needle comprising at least one conduit for receiving a second needle; a connector at the proximal end of the first needle, wherein the connector removably connects the second needle or style to the first needle, and further wherein the first needle comprises a plurality of fenestration at its distal end, and wherein the at least one fenestration is located at an angle of 15 to 30 degrees relative to the longitudinal axis of the first needle and further wherein the at least one fenestration is angled toward the proximal end.

2. The delivery device of claim 1, wherein the connector at the proximal end of the first needle comprises a dual lock connection system.

3. The delivery device of claim 1, wherein the distal end of the second needle or stylet is housed within an opening at the proximal end of the first needle.

4. The delivery device of claim 1, wherein the first needle comprises a triangle opening at the tip of the distal end.

5. The delivery device of claim 4, wherein the triangle opening houses the tip of the second needle.

6. The delivery device of claim 4, wherein the triangle opening comprises at least one fenestration on either side of the opening.

7. The delivery device of claim 2, wherein the dual lock connection system has a male luer at its distal end and a female luer at its proximal end, wherein the female luer connects with a male luer of the second needle.

8. A kit for the delivery of therapeutics wherein the kit comprises a therapeutic delivery device in accordance with claim 1.
